(19) United States
(12) Patent Application Publication
(10) Pub. No.: US 2006/0247634 A1
(43) Pub. Date: Nov. 2, 2006

(54) Spinous Process Spacer Implant and Technique

(76) Inventors: Kenneth D. Warner, Wilton, CT (US); Steven B. Goldstein, Plainview, NY (US)

Correspondence Address: LAURA N. TUNNELL, P.O. BOX 91929, SANTA BARBARA, CA, 93190, US

(21) Appl. No.: 11/308,767

(22) Filed: May 1, 2006

Related U.S. Application Data
(60) Provisional application No. 60/676,538, filed on May 2, 2005.

(51) Int. Cl. A61F 2/30

(52) U.S. Cl. 606/61

(57) ABSTRACT

A spinous process spacer implant for relief of back pain is disclosed. The implant, a generally H-shaped device having grooved ends, is made of human allograft or other suitable artificial or natural material. It is sized for implantation between and optional attachment to adjacent spinous processes. The implant may be hollow in the center to allow for addition of suitable biological material.

This invention generally relates to a device and method for the treatment of spinal stenosis.

### BACKGROUND

Spinal stenosis is a narrowing of the spinal canal. While this in itself does not necessarily cause symptoms, swelling and nerve inflammation results when the narrowing leads to compression of the spinal cord and nerve roots.

While spinal stenosis can be found in any part of the spine, the lumbar and cervical areas are most commonly affected. Patients with lumber spinal stenosis may feel pain, weakness, or numbness in the lower extremities. Symptoms often increase when walking short distances and decrease when the patient sits, bends forward or lies down. Although some people are born with spinal stenosis, it generally occurs as the gradual result of “wear and tear” on the spine during everyday activities, primarily affecting people over 50 years of age.

Non-surgical treatments of spinal stenosis include medication, steroid injections, and physical therapy. While surgical options are available, they are invasive. Due to the inherent risks involved with such procedures, surgery is usually considered only after other non-invasive procedures have failed.

Published application number 2001/0039452 by Zucherman discloses a spinal distraction implant that alleviates pain associated with spinal stenosis by expanding the volume in the spinal canal or neural foramen. In the Zucherman device, a body portion is adapted to seat between the adjacent spinous processes while a wing portion is adapted to prevent lateral movement of the body portion, thereby holding it in place between the adjacent spinous processes.

Although the Zucherman device achieves spinal distraction, it nonetheless presents some limitations. It is a non-biological, multi-piece design, subject to wear and implantation complexity. Furthermore, the expansive geometry of the device may not lend itself to minimally invasive surgical techniques seeking to conserve muscle mass and soft tissue in the regions adjacent the spinous processes.

### SUMMARY

A spinous process spacer device for surgical implantation between the spinous processes of adjacent upper and lower vertebrae is disclosed. The spacer device maintains a desired space between the adjacent spinous processes. It comprises a tubular member having an axis, a length, an axial lumen coextensive with the length, an outer diameter, an upper end and a lower end. The upper and lower ends each have a pair of diametrically opposed notches (cut outs) along the outer diameter of the spacer device. The pair of diametrically opposed notches in the upper end is aligned with the pair of diametrically opposed notches in the lower end. When properly positioned, the pairs of diametrically opposed notches are dimensioned to receive a portion of the spinous processes, thereby maintaining the desired space between adjacent spinal processes.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### DESCRIPTION OF THE PREFERRED EMBODIMENTS

The term “allograft”, as used herein, is intended to mean a graft taken from a different individual of the same species.

The term “sagittal plane”, as used herein, is the plane which splits the body into left and right segments. The mid-sagittal, or median plane splits the body into equal left and right halves.

The term “coronal plane”, as used herein, is the plane that separates the body into anterior and posterior (front and back) segments. The coronal plane is perpendicular to the sagittal plane.

The term “posterior process fusion”, as used herein, describes the fusion of adjacent spinous processes firstly to the spinous process spacer implant, and eventually to each other via the growth of tissue through the axial conduit of the implant.

### DESCRIPTION OF THE FIGURES

FIG. 1 illustrates a spinal column having a collapsed disc and stenotic central canal.

FIG. 2 illustrates the spinous process spacer implant of the present invention.

FIG. 3a illustrates the final configuration of spinous process spacer positioned between adjacent spinous processes. FIG. 3b is an enhancement indicating the engagement of the spinous processes with the spinous process cutout portion of the implant.

FIG. 4 illustrates the spinous process spacer positioned and constrained with optional cables.

FIG. 5 illustrates the introduction of successively larger diameter dilators until the appropriate distraction is achieved.

FIG. 6 illustrates the insertion and placement of the rotation cannula over the last dilator used.

FIG. 7 illustrates the position of the rotation cannula between adjacent spinous processes after the dilators have been removed.

FIG. 8 illustrates the insertion and initial placement of the implant with the rotation instrument.

FIG. 9 illustrates the configuration upon rotating the implant 90 degrees using the rotation instrument.

FIG. 10 illustrates the final configuration of the implant upon removal of the cannula and rotation instrument.

FIG. 11 illustrates the empty rotation cannula.

FIG. 12 illustrates the rotation cannula with the implant inserted into the extensions for the lateral spinous process stabilizers.

FIG. 13 illustrates the rotation instrument.

FIG. 14 illustrates the rotation instrument with the implant inserted between its prongs.

FIG. 15 illustrates the rasping device.

### DESCRIPTION OF NUMERALS USED IN THE FIGURES

10—spinal cord

11—vertebral body

12—spinous process

13—normal interspinous process space

14—collapsed interspinous process space

15—stenotic central canal

16—normal disc

17—collapsed disc

18—anterior side

19—posterior side

20—implant, spinous process spacer

21—tubular member

22—axial conduit

23—spinous process cutout, notch

24—lateral spinous process stabilizers

25—holes for securement means

26—desired interspinous process spatial distance

40—means for securement to spinous processes

50—dilators

60—rotation cannula

80—rotation instrument

110—extension for lateral spinous process stabilizers

111—rotation and removal grooves

112—tapered leading edge of the extension

113—open trailing edge of the extension

130—prong

131—saddle

150—trial rasp

151—bullet shaped leading edge

152—implant shaped body

153—stem

154—handle

155—rasping surface

### Detailed Description of the Preferred Embodiments

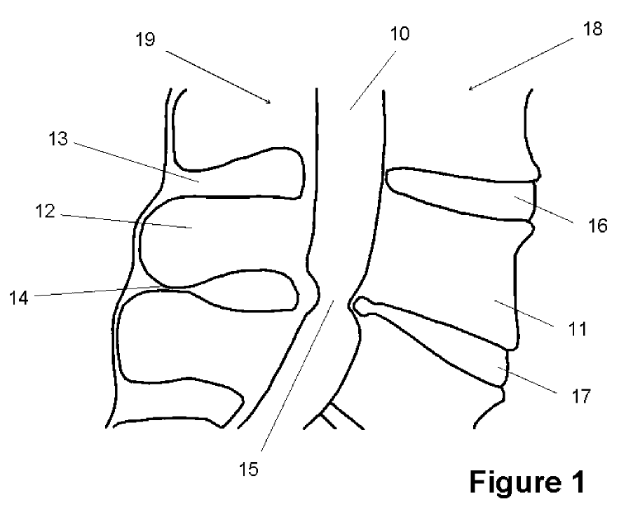

The function of the spinous process spacer (20) can be understood by appreciating the problem illustrated in FIG. 1. A cross section of the spinal cord (10) in the mid-sagittal plane is shown with the vertebral body (11) on the right and the spinous process (12) on the left. A normal disc (16) above (or cephalad to) the vertebral body (11) is shown pairing with a normal interspinous process space (13). In the same manner, a collapsed disc (17) pairs with a collapsed interspinous process space (14). This unbalanced arrangement results in one or more types of narrowing such as the stenotic central canal shown at (15). The spinous process spacer (20) spreads the spinous processes (12) adjacent to the collapsed interspinous process space (14) apart, thereby restoring anatomical alignment of the anterior and posterior spinal anatomy and alleviating the narrowing of nerve pathways that may have been generating severe pain and loss of function.

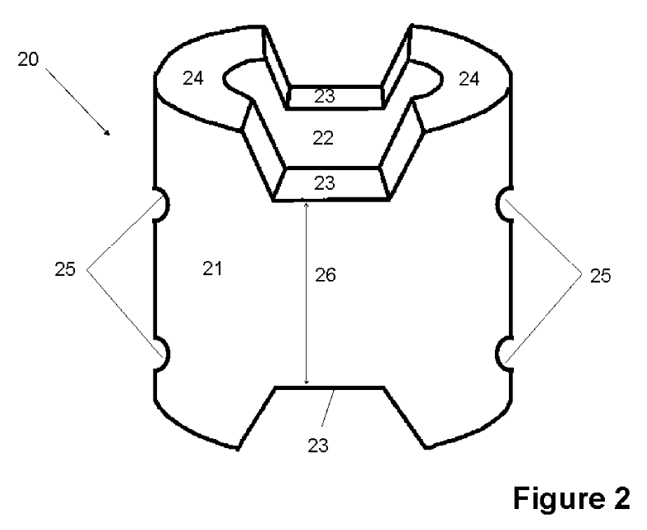

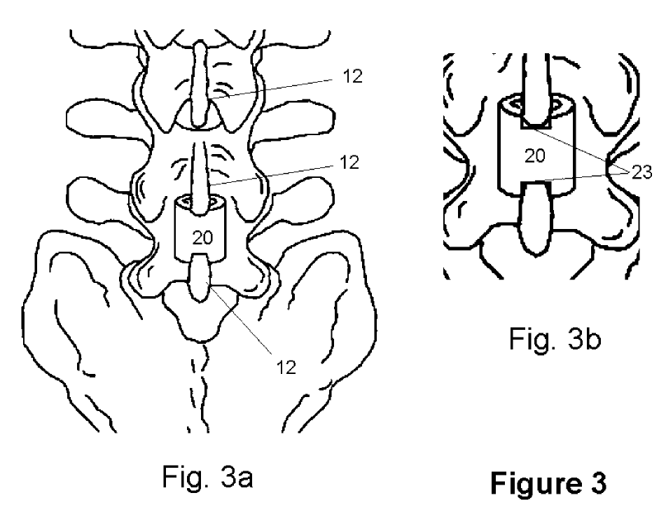

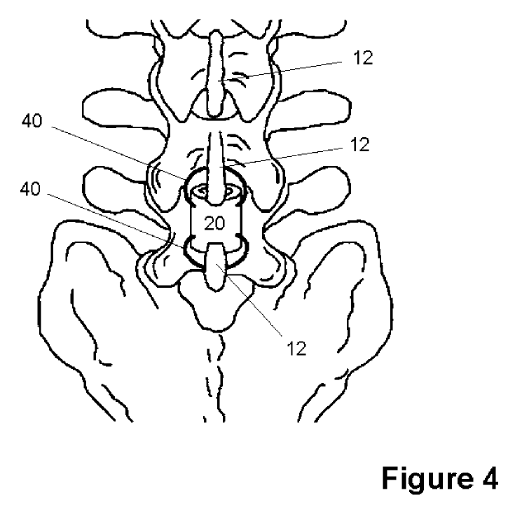

The spinous process spacer (20) of the present invention is shown in FIG. 2. Basically a tubular member (21) having an axial conduit (22), the desired interspinous process space (26) is defined by the depth of diametrically opposed spinous process cutouts, or, more simply termed “notches” (23), cut out of each end. The spinous processes (12) seat in these notches (23) when the spinous process spacer (20) is deployed in its final position as illustrated in FIGS. 3-4. The remaining tubular sections flanking the notches (23) function as lateral spinous process stabilizers (24). Optional holes (25) for mechanical attachment (40) to the adjacent spinous processes (12) are provided as well. Attachment can be effected by suturing, cabling or other suitable means as is indicated in FIG. 4.

The tubular geometry of the spinous process spacer (20) not only serves to strengthen the spacer in the axial direction but also provides more stabilization against unintended rotation than the substantially flat “H” shaped designs of the prior art. Moreover, the axial conduit (22) offers a fillable space for bone growth-promoting materials. Finally, the spinous process spacer (20) can be made of allograft or other suitable biological material to further promote integration of the spinous process spacer (20) into surrounding tissue.

There are many techniques suitable for deployment of the spinal process spacer (20), the choice of which is dependent upon individual circumstances. Basically, the following steps must be executed. The collapsed interspinous process space (14) must be adequately distracted. An appropriately sized spinous process spacer implant (20) must be secured in an implant holder. The holder and implant must be placed within the distracted space so that the axis of the spinous process spacer implant (20) is parallel to the adjacent spinous processes. Finally the spinous process spacer implant (20) is rotated 90 degrees in the mid-sagittal plane so that its axis is now perpendicular to the spinous processes (12), and positioned so that the spinous processes (12) rest within the notches or, alternatively, the spinous process cutouts (23), and engagement is effected. A major benefit of this technique is that it gains access to the spinous process space via a lateral incision in the spinous process ligament. This preserves more of the spinous process ligament than a direct posterior approach.

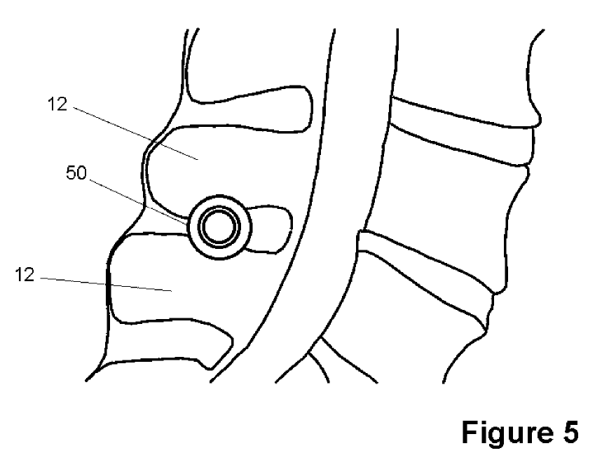

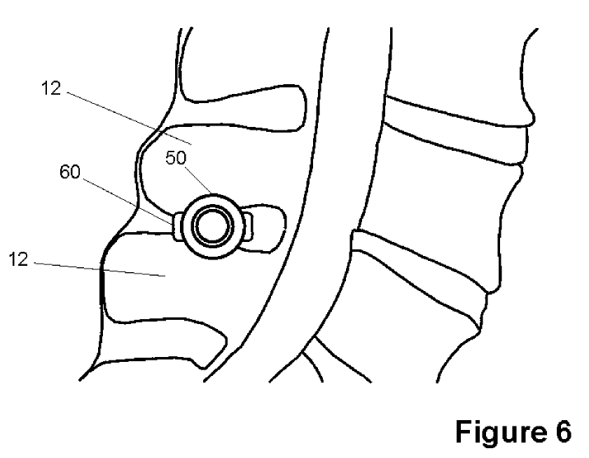

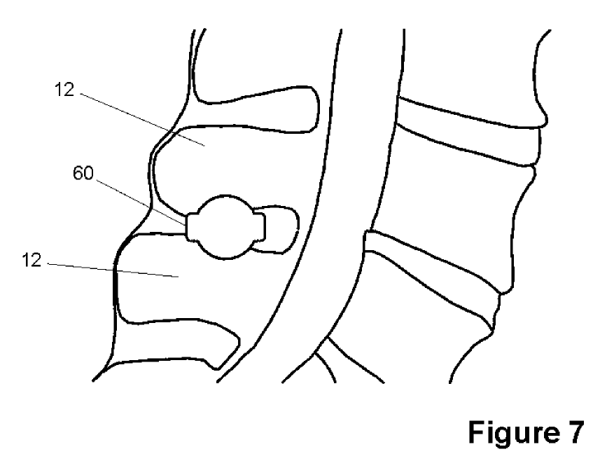

A more detailed description of such a method is illustrated in the remaining FIGS. 5-15. FIG. 5 illustrates a cross section of the mid-sagittal plane wherein dilators (50) have been placed between adjacent spinous processes (12). A rotation cannula (60) is placed over the largest dilator as shown in FIG. 6. The rotation cannula (60) is described in more detail in later paragraphs, but for the present discussion, suffice it to say that the rotation cannula (60) effects the above outlined steps of providing an implant holder and facilitating the required 90 degree rotation. FIG. 7 illustrates the position of the rotation cannula between adjacent spinous processes after the dilators have been removed.

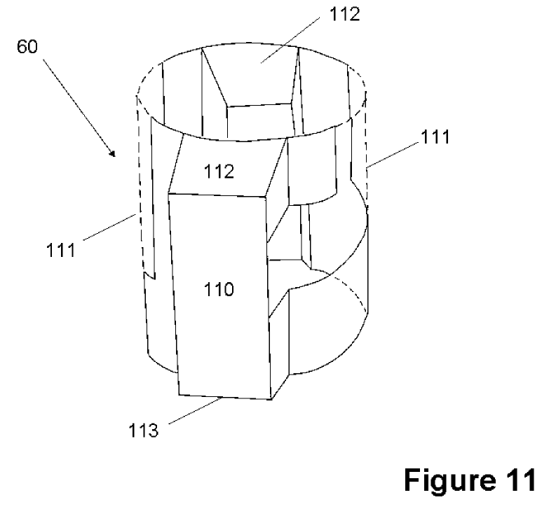

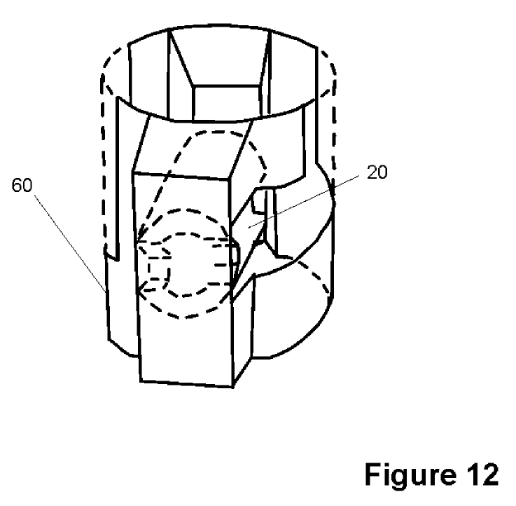

As shown in FIGS. 11-12, the rotation cannula (60) is substantially an open cylinder having a hollow rectangular cross member or, alternatively, an extension for the lateral spinous process stabilizers (110), wherein the spinous process spacer (20) is placed. The leading edge (112) of the cross member (110) is tapered to facilitate initial placement between the spinous processes (12). The trailing edge (113) is open.

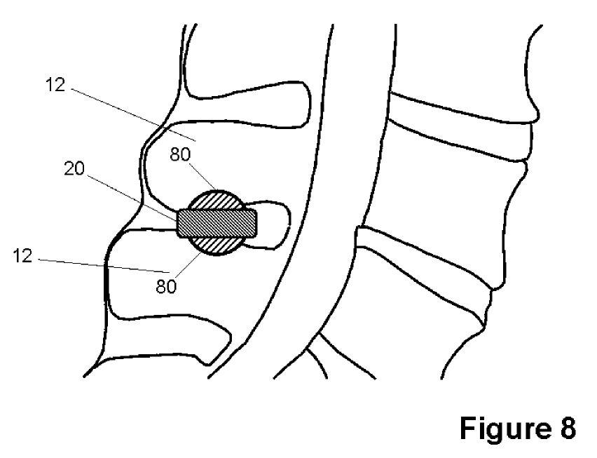

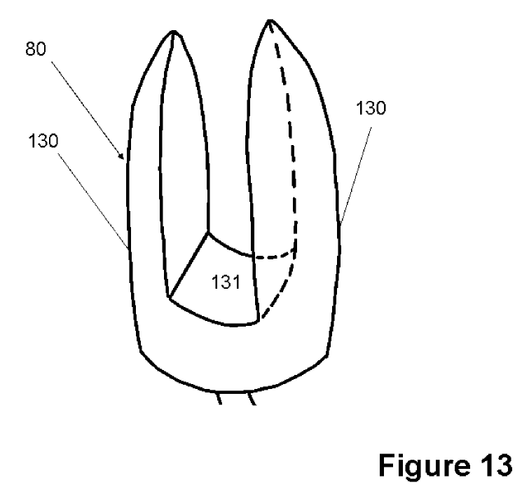

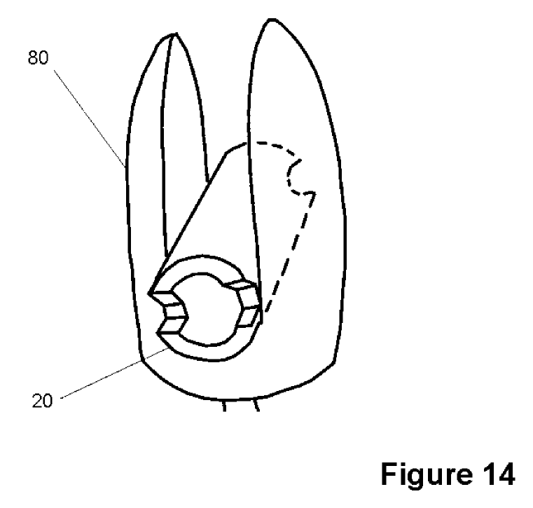

The long dimension of the rectangular cross member (110) is placed parallel to the affected spinous processes (12). With the spinous process spacer (20) saddled (131) between its prongs (130) as shown in FIGS. 13-14, the rotation instrument (80) effects placement of the spinous process spacer (20) in the rotation cannula (60) as is shown in FIG. 8. A cross section of each prong (130) flanking the spinous process spacer (20) can be seen in the figure.

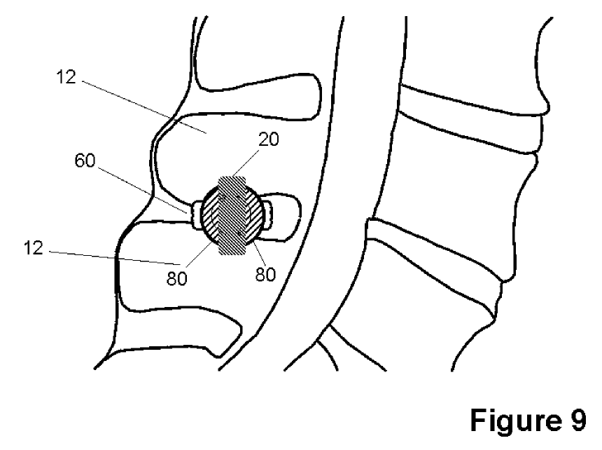

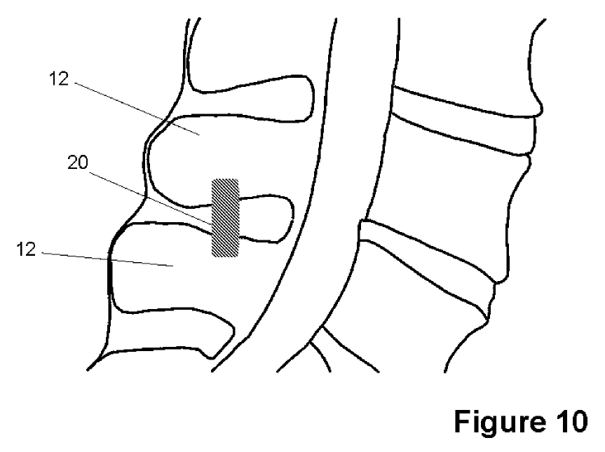

The rotation cannula (60), shown in more detail in FIGS. 11-12, is designed so that various grooves (111) allow the 90 degree rotation of the spinous process spacer (20) and removal of the rotation cannula (60)/rotation instrument (80) combination leaving only the spinous process spacer (20) in place. FIG. 9 illustrates the resulting configuration after the required 90 degree rotation has been performed. Finally, FIG. 10 illustrates the placement of the spinous process spacer (20) upon removal of the cannula and rotation instrument. This lateral entry to the spinous process space combined with the 90 degree rotation of the spinous process spacer to its final position, will allow for greater preservation of the spinous process ligament and surrounding soft tissue anatomy than if the surgical approach is from a direct posterior approach.

One of the most unique features of this invention is the fusion promoting features of its design, most particularly its axial conduit (22) which not only provides a pathway wherein fusion can occur, but also provides a fillable space wherein fusion-promoting biological material can be deployed. To further this end, an additional step in the deployment technique can be used.

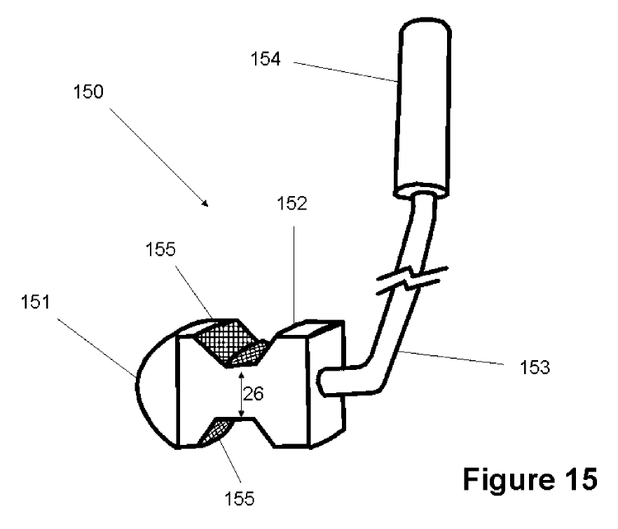

The trial rasp (150), shown in FIG. 15, is introduced into the interspinous process space (14) after distraction. Having a bullet shaped leading edge (151) to facilitate insertion, its body (152) replicates the shape of the spinous process spacer (20), particularly the dimension matching the desired interspinous process spatial distance (26).

The trial rasp (150) accomplishes several things. It tests the space for acceptance of the intended implant (20) by placing a trial in place which is representative of the size and shape of the actual implant (20). Introduction of the trial rasp (150) and particularly the rasping surface (155) causes bleeding bone and enhances bone growth and fusion. It additionally shapes and prepares the affected area of the spinous process (12) to engage and mate more intimately with the notch (23). Furthermore, it strips away soft tissue from the engagement area that might otherwise be caught between the implant (20) and the spinous process (12), thereby inhibiting fusion

Fusion resulting between the spinous process spacer implant and the two neighboring spinous processes (12), will be much less rigid than the traditional, more invasive, posterior lateral fusion or an interbody fusion. This lessened rigidity will serve to pose less risk upon the the adjacent spine motion segment and consequently less risk of a condition known as “adjacent segment disease”, a serious side effect of more traditional methods.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A spinous process spacer device operable for surgical implantation between the spinous processes of adjacent upper and lower vertebrae, the spacer device thereafter maintaining a desired space between the adjacent spinous processes, the spacer device comprising a tubular member having an axis, a length, an axial lumen coextensive with said length, an outer diameter, an upper end and a lower end, said upper and lower ends each having a pair of diametrically opposed notches therein along said outer diameter of said spacer device wherein said pair of diametrically opposed notches in said upper end is aligned with said pair of diametrically opposed notches in said lower end and wherein said pairs of diametrically opposed notches are dimensioned to receive a portion of said spinous processes therewithin and said axial lumen providing a pathway for fusion of said spinous processes of said adjacent upper and lower vertebrae when said spacer device is disposed between said adjacent spinous processes.

2. The spinous process spacer device of claim 1 wherein said spinous process spacer device is made from a biocompatible material.

3. The spinous process spacer device of claim 1 further comprising attachment means operable for attachment of said spinous process spacer device to said adjacent spinous processes.

4. The spinous process spacer device of claim 2 further comprising attachment means operable for attachment of said spinous process spacer device to said adjacent spinous processes.

5. A method for creating and maintaining a desired space between adjacent spinous processes comprising the steps of:
surgically exposing said adjacent spinous processes; then
elevating said adjacent spinous processes to create a space therebetween equal to or greater than said desired space; then
presenting a spinous process spacer device in accordance with claim 1,
placing the device between said adjacent spinal processes such that said axis of said tubular member is substantially at a right angle with respect to said spine,
rotating said device 90 degrees within the mid-sagital plane such that said axis of said tubular member is substantially parallel to said spine and,
positioning said pairs of said notches such that said portion of said spinous process are engaged therewithin, effecting the maintenance of said desired space between said adjacent spinous processes.

6. A method for creating and maintaining a desired space between adjacent spinous processes comprising the steps of:
surgically exposing said adjacent spinous processes; then
elevating said adjacent spinous processes to create a space therebetween equal to or greater than said desired space; then
presenting a spinous process spacer device in accordance with claim 2,
placing the device between said adjacent spinal processes such that said axis of said tubular member is substantially at a right angle with respect to said spine,
rotating said device 90 degrees within the mid-sagital plane such that said axis of said tubular member is substantially parallel to said spine and,
positioning said pairs of said notches such that said portion of said spinous process are engaged therewithin, effecting the maintenance of said desired space between said adjacent spinous processes.

7. A method for creating and maintaining a desired space between adjacent spinous processes comprising the steps of:
surgically exposing said adjacent spinous processes; then
elevating said adjacent spinous processes to create a space therebetween equal to or greater than said desired space; then
presenting a spinous process spacer device in accordance with claim 3,
placing the device between said adjacent spinal processes such that said axis of said tubular member is substantially at a right angle with respect to said spine,
rotating said device 90 degrees within the mid-sagital plane such that said axis of said tubular member is substantially parallel to said spine and,
positioning said pairs of said notches such that said portion of said spinous process are engaged therewithin, effecting the maintenance of said desired space between said adjacent spinous processes.

8. A method for creating and maintaining a desired space between adjacent spinous processes comprising the steps of:
surgically exposing said adjacent spinous processes; then
elevating said adjacent spinous processes to create a space therebetween equal to or greater than said desired space; then
presenting a spinous process spacer device in accordance with claim 4,
placing the device between said adjacent spinal processes such that said axis of said tubular member is substantially at a right angle with respect to said spine,
rotating said device 90 degrees within the mid-sagital plane such that said axis of said tubular member is substantially parallel to said spine and,
positioning said pairs of said notches such that said portion of said spinous process are engaged therewithin, effecting the maintenance of said desired space between said adjacent spinous processes.
